(19) United States
(12) Patent Application Publication
(10) Pub. No.: US 2022/0383990 A1
(43) Pub. Date: Dec. 1, 2022

(54) System and Method to Predict Mass Transport from Complex Release Systems Using Experimental Data-based Modeling

(71) Applicant: DigiM Solution LLC, Winchester, MA (US)

(72) Inventor: Shuang Zhang, Winchester, MA (US)

(21) Appl. No.: 17/804,355

(22) Filed: May 27, 2022

Related U.S. Application Data
(60) Provisional application No. 63/194,656, filed on May 28, 2021.

(51) Int. Cl. G16C 20/30; G16C 20/70

(52) U.S. Cl. CPC G16C 20/30; G16C 20/70

(57) ABSTRACT

Embodiments determine models that predict release profiles of substances from material matrices. An embodiment constructs a simulation model of a release system based on experimental data such as imaging and determines a model predicting a release profile through use of an iterative process. The process iterates: (i) modifying parameters of the constructed simulation model based upon release system mechanistic characteristic data to correct a transport coefficient of the release system and (ii) performing a simulation of the release system using the constructed simulation model with the modified parameters to generate a simulation-based release profile, until a given simulation-based release profile that matches the release system characteristic data is identified. The constructed simulation model with the modified parameters used to generate the matching simulation-based release profile is set as the model predicting the release profile of the substance from the material matrix.

### RELATED APPLICATION

This application claims the benefit of U.S. Provisional Application No. 63/194,656, filed on May 28, 2021. The entire teachings of the above application are incorporated herein by reference.

### BACKGROUND

Controlling the rate of a material substance transported out of a material matrix has wide applications, including controlled release of active pharmaceutical ingredients (API), controlled release of insecticide in agriculture, and slow release of anti-corrosion agents, amongst other examples. In such mass transport rate-controlled systems, one or more material substances (referred to as substance hereafter), e.g., API, is often encapsulated in a material matrix (referred to as matrix hereafter), which is composed of one or more materials different from the substances.

### SUMMARY

The design, manufacture, and utilization of rate-controlled systems, i.e., release systems, often relies on simulation and modeling to predict behavior, e.g., performance, of the release systems. While methods and systems exist for simulating release systems and predicting release system behavior, the existing methods could benefit from both accuracy improvements and broadened applicability.

In one such existing method, Zhang, U.S. Pat. No. 11,081,212 B2, entitled “SYSTEM AND METHOD FOR COMPUTING DRUG CONTROLLED RELEASE PERFORMANCE USING IMAGES,” a mass transport profile (e.g., the release profile of a substance, such as API) is predicted from a three-dimensional (3D) digital representation of the release system before it is subjected to a release environment (hereafter referred to as time zero sample, or T0 sample in short). The release of API from a T0 sample is primarily dictated by the network of the API dispersed in a non-API matrix, when the non-API matrix is not changing, i.e., static, during the API release. The release of the API is assumed to be diffusion limited, not solubility limited.

Embodiments of the present invention provide methods and systems that further generalize the functionality described in U.S. Pat. No. 11,081,212. In particular, embodiments utilize additional mathematical models, testing or imaging data, and an iterative validation workflow, to remove assumptions utilized in U.S. Pat. No. 11,081,212, namely, the assumption that the non-API matrix is not changing and the release of the API is diffusion limited. As such, embodiments described herein can be used to predict release profiles for more complex release systems such as bio-degradable implants, pore-closing intrauterine systems, and controlled release of poorly soluble drugs.

One such example embodiment is directed to a method of determining a model that predicts a release profile of a substance from a material matrix. Such an example method starts by constructing a simulation model of a release system based on imaging data of the release system. The release system comprises a material matrix and a substance dispersed in the matrix. To continue, the method determines a model predicting a release profile of the substance from the material matrix through use of an iterative process. In an embodiment, until a given simulation-based release profile matches release system characteristic data, the method iteratively: (i) modifies parameters of the constructed simulation model based upon the release system characteristic data to correct a transport coefficient of the release system and (ii) performs a simulation of the release system using the constructed simulation model with the modified parameters to generate a simulation-based release profile. Once a simulation-based release profile that matches the release system characteristic data is identified, the constructed simulation model with the modified parameters used in performing the simulation to generate the matching simulation-based release profile is set as the model predicting the release profile of the substance from the material matrix.

According to an embodiment, modifying the parameters of the constructed simulation model based upon the release system characteristic data comprises modifying parameters of the constructed simulation model affecting the transport coefficient. In an embodiment, the imaging data is of a representative sample of the release system and the simulation model is constructed based on both (i) the imaging data of the representative sample of the release system and (ii) known parameters approximating a release mechanism of the release system.

An embodiment determines the model predicting the release profile of the substance from the material matrix in an iteration by first, creating a first modified model of the release system. The first modified model is created by modifying, in accordance with the release system characteristic data, given parameters of the constructed simulation model to modify the transport coefficient due to a difference between actual diffusivity of the substance and diffusivity in the given parameters. Second, in the iteration, a simulation is performed using the first modified model to determine a first simulation-based release profile. In this example, the first simulation-based release profile is the given simulation-based release profile that matches the release system characteristic data.

Embodiments of the method may perform any number of iterations to determine the model predicting the release profile of the substance. For instance, an example implementation includes a first iteration in which a first modified model of the release system is created by modifying, in accordance with the release system characteristic data, given parameters of the constructed simulation model to modify the transport coefficient due to a difference between actual diffusivity of the substance and diffusivity in the given parameters. In this first iteration, a simulation is performed using the first modified model to determine a first simulation-based release profile. In this example, the first simulation-based release profile does not match the release system characteristic data. The method then moves to a second iteration where a second modified model of the release system is created. The second modified model is created by modifying, in accordance with the release system characteristic data, given parameters of the first modified model to correct changes to the transport coefficient due to variability in diffusivity of the substance over time caused by changes of the material matrix during the release. In this second iteration, a simulation is performed using the second modified model to determine a second simulation-based release profile. For this example, the second simulation-based release profile is the given simulation-based release profile that matches the release system characteristic data.

Another embodiment constructs the simulation model based upon both the imaging data and known parameters that approximate a release mechanism of the release system. According to an embodiment, the release system characteristic data comprises in vitro release system characteristic data. In such an embodiment, determining a model predicting the release profile of the substance from the material matrix comprises, until the given simulation-based release profile matches the in vitro release system characteristic data, iteratively (i) modifying the parameters of the constructed simulation model affecting the transport coefficient in accordance with the in vitro release system characteristic data and (ii) performing a simulation of the release system using the constructed simulation model with the modified parameters to generate a simulation-based release profile.

Another example embodiment performs a fitting (e.g., least squares) between the given simulation-based release profile and a release profile indicated by the release system characteristic data to determine if the given simulation-based release profile matches the release system characteristic data.

According to an embodiment, the release system characteristic data includes at least one of: images of the release system at multiple points in time; in vitro data of the release system (including substance and matrix, e.g., degradable matrix, making up the release system); in vivo data of the release system (including substance and matrix, e.g., degradable matrix, making up the release system); a polymer model derived experimentally or empirically; an empirical release model; an indication of geometry of the release system being cylindrical, spherical, or plate; membrane coating dimensions; an indication of substance transport being substance evacuated pore diffusion, substance evacuated pore diffusion plus polymer diffusion, or osmotic pump; an indication of pre-existing porosity; an indication of polymer transport as surface erosion or matrix erosion; an indication of polymer dislocation as swelling caused pore closure, swelling caused matrix diffusion, or auto-hydrolysis; drug concentration; bulk diffusivity; drug solubility; flow rate; pH dependency; and temperature dependency.

In embodiments, the system may be any substance or combination of substances. For example, in an embodiment the substance is at least one of: a pharmaceutical ingredient; an insecticide; and an anti-corrosion agent, amongst other examples. According to an example embodiment, the material matrix is a polymer. In one such embodiment, the polymer is either biostable or biodegradable.

In an embodiment, constructing the simulation model of the release system based on the imaging data of the release system includes determining at least one image-derived release system characteristic from the imaging data. In turn, a parameter of the simulation model of the release system is updated to correspond to the determined at least one image-derived release system characteristic. In embodiments, image-derived release system characteristics may include: size of the substance, amount of the substance, location of the substance in the release system, size of the material matrix, amount of the material matrix, location of the material matrix, porosity of the material matrix, pore size of the material matrix, location of pores, fracture size, fracture location, size of an additive in the release system, amount of the additive, location of the additive in the release system, and evolution of any such image-derived release system characteristics over time, amongst other examples.

According to an embodiment, the transport coefficient indicates at least one of: diffusivity of the substance through the release system; diffusivity of the substance over time caused by changes of the release system during the release; permeability of the substance through the release system; and permeability of the substance over time caused by changes of the release system during the release. In an embodiment, the changes of the release system include at least one of: phase transformation of the substance; phase transformation of the matrix; polymorph transformation of the substance; polymorph transformation of the matrix; degradation of the matrix; surface erosion of the matrix; bulk erosion of the matrix; matrix swelling; matrix deformation; matrix dislocation; pore forming; pore closure; fractures; hydrolysis; relocation of the substance; relocation of the matrix, aggregation of the substance; aggregation of the matrix; dispersion of the substance; and dispersion of the matrix.

Another embodiment is directed to a system that includes a processor and a memory with computer code instructions stored thereon. In such an embodiment, the processor and the memory, with the computer code instructions, are configured to cause the system to implement any embodiments or combination of embodiments described herein.

Yet another embodiment is directed to a computer program product for determining a model predicting a release profile of a substance from a material matrix. The computer program product comprises one or more non-transitory computer-readable storage devices and program instructions stored on at least one of the one or more storage devices. The program instructions, when loaded and executed by a processor, cause an apparatus associated with the processor to perform any embodiments or combination of embodiments described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a visual depiction of factors that influence substance release that may be modeled using embodiments.

FIG. 2 is a flowchart of a method to determine a release profile of a substance from a material according to an embodiment.

FIG. 3 is an iterative method to determine a release profile that is employed in the method of FIG. 2 according to an embodiment.

FIG. 4 is a block diagram of an embodiment of a system to iteratively predict release profiles.

FIG. 5 is a workflow diagram of an embodiment of an iterative data-based release prediction method.

FIG. 6A shows a cross-section of three-dimensional images of a microsphere sample acquired via focused ion beam scanning electron microscopy (FIB-SEM) in an embodiment.

FIG. 6B depicts a reconstructed API network.

FIG. 6C illustrates a reconstructed porosity network.

FIG. 7 is a depiction of a software architecture that may be utilized by embodiments.

FIG. 8 illustrates a computer network, or similar digital processing environment, in which embodiments of the present invention may be implemented.

FIG. 9 is a diagram of an example internal structure of a computer (e.g., client processor/device or server computers) in the computer system of FIG. 8.

### DETAILED DESCRIPTION

A description of example embodiments follows.

Embodiments provide improved methodologies for determining models that predict release profiles of substances from material matrices. As noted above, existing methods, such as those described in Zhang, U.S. Pat. No. 11,081,212 B2, assume that the matrix is not changing and the release of the substance is diffusion limited. Embodiments of the present invention do not rely on these assumptions and, as a result, can be used to predict release profiles for more complex release systems such as bio-degradable implants, pore-closing intrauterine systems, and controlled release of poorly soluble drugs.

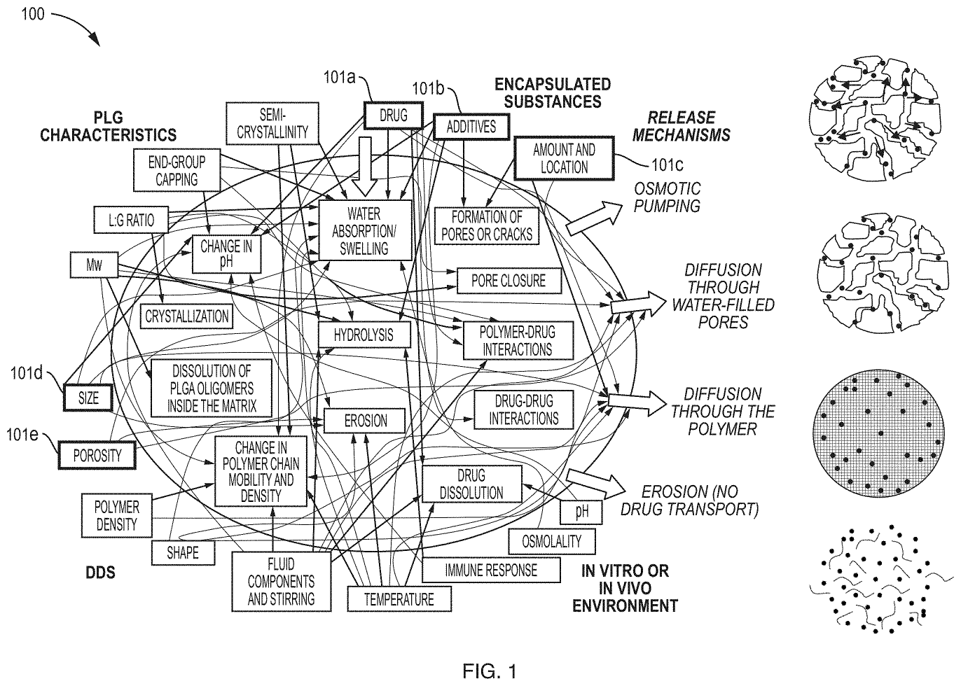

When the static matrix assumption is removed, mass transport can be very complex. FIG. 1 shows a release system 100 and the factors of parameters, e.g., 101a-d, that influence API release of drug products with drug particles dispersed into a poly (D, L-lactic-co-glycolic acid) (PLGA) polymer matrix. PLGA is a representative matrix that has a complex response to release media. As soon as the non-API matrix starts to contribute to release, the release system becomes complex. For example, such complex behavior includes the dynamic change of polymer, functional excipients, porosity, microscopic API solubility localization, or any combination of the above, amongst other examples. The complex release behavior can contribute significantly to the drug release, particularly for long acting and extended-release drug products. In some cases, the complex behavior can over take the API network as the dominant release mechanism. For example, when drug loading is low in some intrauterine devices for multi-year birth control, the API does not form a percolating network. Hence, the drug release is pre-dominantly through polymer swelling and subsequent drug diffusion through the swelled polymer.

When the diffusion-limited substance, e.g., API, assumption is removed, solubility of the substance is not instantaneous. As such, release rate is influenced by how fast the substance dissolves into the media and how fast the dissolved substance is transported out of the release system. The rate of dissolution and transport can compete, leading to a potentially dynamic transition from a transport-limited release system to a dissolution-limited system.

Embodiments of the invention generalize the functionality described in Zhang, U.S. Pat. No. 11,081,212 B2 to applications where the aforementioned two assumptions are removed. Hereinbelow, the first section describes a general methodology to study complex release systems using physical testing data and image-based release modeling. In comparison with the release modeling methods in the literature, the use of images of T0 samples is a key differentiator. The second section describes an embodiment of the iterative method with three iterations.

1. An Iterative Dissection and Validation Methodology to Predict Complex Drug Release by Combining Physical Testing Data with Image-Based Release Modeling

The complexity of release of the main substance, e.g., API, from a matrix can be elucidated using a pharmaceutical drug eluding implant example. API (an example main substance) is often dispersed into a polymer matrix, such as PLGA. PLGA itself has wide applications. In addition to controlled release of encapsulated small molecule drugs, PLGA can be used for tissue engineering (Oh and Lee, 2007; Wang et al., 2010), healing of bone defects (Bertoldi et al., 2008), vaccines (Feng et al., 2006; Jiang et al., 2005), biopharmaceuticals such as proteins and peptides, and hydrophobic drugs with low oral bioavailability (Närhi and Nordstrom, 2005; Pisal et al., 2010; Wiscke and Schwendeman, 2008), amongst other examples. When a polymer matrix, such as PLGA, is loaded with a drug substance, the functionality described herein can be applied to determine a model predicting a release profile of the drug substance from the matrix. For drugs unsuitable for oral admission due to low oral bioavailability, patient compliance is also low due to the necessity of administration by injection. The frequency of injections can be decreased through the use of controlled-release delivery system, which is very beneficial for patients who require daily and/or long-term treatment.

The reasons for the widespread use of PLGA are its biodegradability, its biocompatibility, and the fact that drug products containing PLGA have been approved for parenteral use by regulatory authorities around the world. Further advantages of PLGAs are that they are commercially available with very different physicochemical properties, and that the drug release profile can be tailored by selecting PLGAs with the appropriate properties, for example, molecular weight (Mw) and lactide:glycolide ratio (L:G). The duration of drug release can be varied from hours to months. Furthermore, pulsed drug release is also possible. Blending or co-polymerizing PLGA with other materials, or encapsulating PLGA microparticles in gels, further extends the possibility of controlling drug release. PLGA implants may be surgically inserted at a desired location, providing the advantage of local drug delivery of, for example, antibiotics or anti-cancer drugs.

Knowledge of the release mechanisms and the physicochemical processes that influence the release rate is vital in order to develop controlled-release systems. There are primarily four possible release mechanisms, based on types of transport (transport driven or degradation/erosion driven) and the media that transport is permitted. The four possible release mechanisms are (1) diffusion through water-filled pores, (2) diffusion through the polymer, (3) osmotic pumping, and (4) erosion (i.e., no drug transport).

The release rate of a PLGA-based release system is often said to be diffusion-controlled initially and degradation/erosion controlled during a final stage of the release period. However, many processes or events influence the rate of drug diffusion and the degradation kinetics, for example, polymer-drug interactions, drug-drug interactions, water absorption, and pore closure. Knowledge regarding these more detailed processes is necessary to be able to understand the drug release in detail and to be able to control the release rate. Drug release is often preceded by a chain of processes (e.g., water absorption, hydrolysis, and erosion). These processes are influenced by many different factors. This further increases the complexity of drug release.

While PLGA represents a commercially popular and regulatorily proven matrix material, other microstructure features that can influence release, include: (1) pre-existing porosity and the porosity's evolution over time, (2) diffusion rate change through the polymer matrix during the release, (3) hydrolysis of drug, polymer, or a third excipient, (4) polymer deformation due to temperature, pH, and other release environment parameters, (5) polymer deformation due to residual stress relaxation from molding, extrusion, and other manufacturing processes, (6) fracturing, and (7) reduction of drug solubility locally due to the presence of porous polymer matrix.

FIG. 1, extracted from the review paper Fredenberga et. al., 2011, illustrates the complexity of the release process of a drug delivery system 100 where API is encapsulated with PLGA polymer. FIG. 1 shows a system 100 where different factors e.g., 101a-e, influence drug release from a PLGA matrix. The effects of the properties of the drug delivery system and the surrounding environment of the processes that, in turn, influence drug release are illustrated by the arrows. While the illustration of the system 100 is helpful to identify the potential influencing parameters and their interactions, it is impractical to model all these parameters at once. A great number of modeling approaches have been published (Arifin et. al., 2006, Siepmann & Gopferich 2001, Siepmann & Siepmann 2008, and Fredenberga et. al., 2011). However, a common deficiency in these published approaches is that drug-polymer-porosity microstructure is either ignored, or homogenized into one or several parameters where the microstructure details are lost.

Image-based modeling approaches, such as those described in U.S. Pat. Nos. 11,081,212, 10,830,713 B2, and U.S. Patent Publication No. 2021/0304391 A1 take microstructure arrangement in T0 samples into full consideration. Important parameters such as drug 101a, additives to the matrix 101b, their amount and locations 101c, size 101d, and porosity 101e, highlighted in FIG. 1, can be reconstructed from the images of T0 samples (i.e., samples of the release system before any release occurs) without making assumptions regarding the parameters 101a-e.

Imaging data from T0 samples represents one kind of release system experimental data. The combination of experimental data with release modeling offers a unique possibility to dissect complex release systems, understand release mechanisms, and optimize release design.

Computational modeling includes image-based modeling where empirical models and mathematical models can be employed. Some example mathematical models are: exponential models (Mollo and Corrigan, 2003), models based on percolation theory (Batycky et al., 1997; Ehtezazi and Washington, 2000), compartment models (Murty et al., 2004), Monte Carlo simulations (Barat et al., 2008), models based on convolution (Guse et al., 2006a), and Fourier analysis (Raiche and Puleo, 2006).

Below embodiments of iterative methods that incorporate experimental data with computational modeling are described.

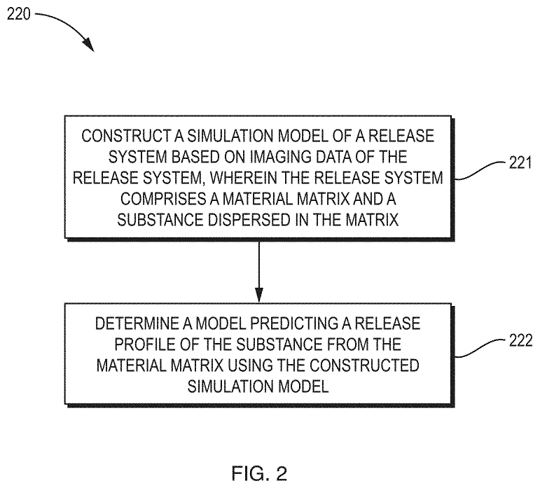

FIG. 2 is a flowchart of a method 220 of determining a model that predicts a release profile of a substance from a material matrix. The method 220 starts at step 221 by constructing a simulation model of a release system based on imaging data of the release system. The release system comprises a material matrix and a substance dispersed in the material matrix. The simulation model was reconstructed from the images of the release system, where the greyscale voxels corresponding to the matrix and those corresponding to the substance dispersed are first segmented into respective material phases.

In an embodiment, the imaging data utilized at step 221 is of a representative sample of the release system and the simulation model is constructed at step 221 based on both (i) the imaging data of the representative sample of the release system and (ii) known parameters approximating a release mechanism of the release system. In an embodiment of the method 220, constructing the simulation model at step 221 includes determining at least one image-derived release system characteristic from the imaging data. At step 221, a parameter of the simulation model of the release system is updated to correspond to the determined at least one image-derived release system characteristic. In embodiments, image-derived release system characteristics include: size, e.g., distribution, of the substance particles, amount of the substance, location of the substance in the release system, size, e.g., distribution, of the material matrix domains, amount of the material matrix, location of the material matrix, porosity of the material matrix, pore size of the material matrix, location of pores, fracture size, fracture location, size distribution of one or more additive particles in the release system, amount of the one or more additives, location of the additives in the release system, and evolution of any such characteristics over time, amongst other examples.

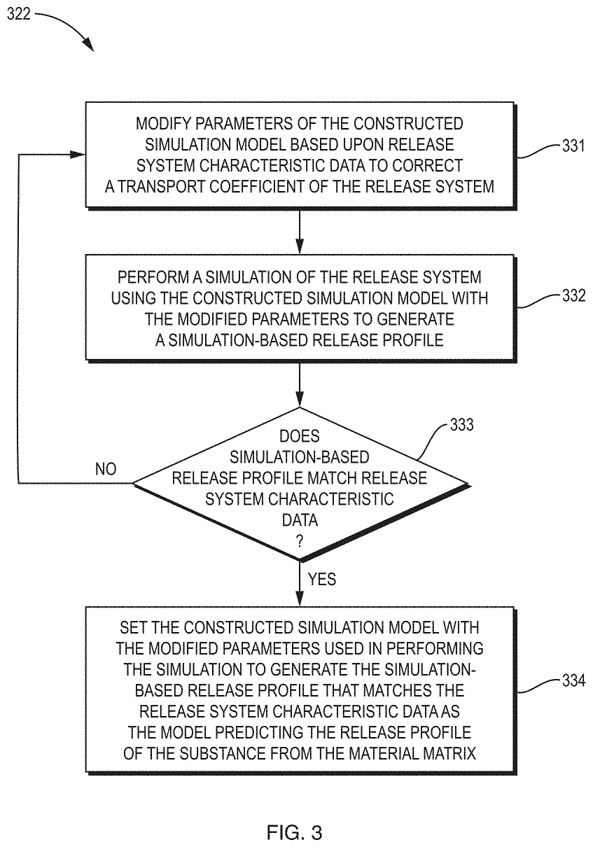

To continue, at step 222, the method 220 determines a model predicting a release profile of the substance from the material matrix using the model that was constructed at step 221. At step 222, the method 220 utilizes the method 322 depicted in FIG. 3 to determine the model.

Turning to FIG. 3, the method 322 iterates steps 331-333 until a given simulation-based release profile matches release system characteristic data. This iterative functionality begins at step 331 by modifying parameters of the constructed simulation model (i.e., the model constructed at step 221 of the method 220) based upon the release system characteristic data. At step 331, the parameters are modified to correct a transport coefficient of the release system. Example transport coefficients are effective diffusivity coefficient and permeability. Effective molecular diffusivity coefficient describes the passive material transport via molecular diffusion. Permeability describes the active material transport via a pressure gradient or flux. According to an embodiment, modifying the parameters of the constructed simulation model based upon the release system characteristic data at step 331 comprises modifying parameters of the constructed simulation model affecting the transport coefficient. In an example embodiment of the method 322, at step 331, a modified model of the release system is created by modifying, in accordance with the release system characteristic data, given parameters of the constructed simulation model to modify error in the transport coefficient due to a difference between actual coefficient of the substance and coefficient in the given parameters used as default.

Next, at step 332 a simulation of the release system is performed using the constructed simulation model with the modified parameters to generate a simulation-based release profile.

At step 333, a check is performed to determine if the simulation-based release profile (generated at step 332) matches the release system characteristic data. If the simulation-based release profile does not match the release system characteristic data, the method moves to step 331 where the method repeats steps 331-333. It is noted that if the method 322 returns to step 331 from step 333, the model modified at step 331 is typically the modified model from the previous iteration.

If at step 333, it is determined that the simulation-based release profile matches the release system characteristic data, the method 322 moves to step 334. At step 334 the constructed simulation model with the modified parameters used at step 332 to perform the simulation that generated the matching simulation-based release profile is set as the model predicting the release profile of the substance from the material matrix.

Embodiments of the method 322 may perform any number of iterations to determine the model predicting the release profile of the substance. To illustrate, in a first iteration an example embodiment creates a first modified model of the release system at step 331 by modifying, in accordance with the release system characteristic data, given parameters of the constructed simulation model to modify the transport coefficient due to a difference between actual diffusivity of the substance and diffusivity in the given parameters. In this first iteration, a simulation is performed at step 332 using the first modified model to determine a first simulation-based release profile. At step 333 it is determined that the first simulation-based release profile does not match the release system characteristic data and the method 322 moves to step 331 and begins a second iteration. At step 331 of the second iteration a second modified model of the release system is created by modifying, in accordance with the release system characteristic data, given parameters of the first modified model to correct changes to the transport coefficient due to variability in diffusivity of the substance over time caused by changes of the material matrix during the release. In this second iteration, a simulation is performed at step 333 using the second modified model to determine a second simulation-based release profile. In this second iteration, the second simulation-based release profile is the given simulation-based release profile, i.e., the simulation-based release profile that matches the release system characteristic data and the method 322 moves to step 334.

According to an embodiment, the release system characteristic data comprises in vitro release system characteristic data. In such an embodiment, determining a model predicting the release profile of the substance from the material matrix (step 222 of the method 220 of FIG. 2) comprises performing the method 322 until, at step 333, it is determined that a simulation-based release profile generated at step 332 matches the in vitro release system characteristic data. In such an embodiment, the modifying at step 331 modifies the parameters of the constructed simulation model affecting the transport coefficient in accordance with the in vitro release system characteristic data.

At step 333 an embodiment determines whether a given simulation-based release profile matches the release system characteristic data by performing a fitting (such as least squares) between the given simulation-based release profile (the simulation profile generated at step 332) and a release profile indicated by the release system characteristic data.

According to an embodiment, the release system characteristic data used at step 331 includes at least one of: images of the release system at multiple points in time; in vitro data of the release system (including substance and matrix, e.g., degradable matrix, making up the release system); in vivo data of the release system (including substance and matrix, e.g., degradable matrix, making up the release system); a polymer model derived experimentally or empirically; an empirical release model; an indication of geometry of the release system being cylindrical, spherical, or plate; membrane coating dimensions; an indication of substance transport being substance evacuated pore diffusion, substance evacuated pore diffusion plus polymer diffusion, or osmotic pump; an indication of pre-existing porosity; an indication of polymer transport as surface erosion or matrix erosion; an indication of polymer dislocation as swelling caused pore closure, swelling caused matrix diffusion, or auto-hydrolysis; drug concentration; bulk diffusivity; drug solubility; flow rate; pH dependency; and temperature dependency.

According to an embodiment of the method 322, the transport coefficient indicates at least one of: diffusivity of the substance through the release system; diffusivity of the substance over time caused by changes of the release system during the release; permeability of the substance through the release system; and permeability of the substance over time caused by changes of the release system during the release. In an embodiment, the changes of the release system include at least one of: phase transformation of the substance; phase transformation of the matrix; polymorph transformation of the substance; polymorph transformation of the matrix; degradation of the matrix; surface erosion of the matrix; bulk erosion of the matrix; matrix swelling; matrix deformation; matrix dislocation; pore forming; pore closure; fractures; hydrolysis; relocation of the substance; relocation of the matrix, aggregation of the substance; aggregation of the matrix; dispersion of the substance; and dispersion of the matrix.

In embodiments, the substance in the release system may be any substance or combination of substances. For example, in an embodiment, the substance is at least one of: a pharmaceutical ingredient; an insecticide; and an anti-corrosion agent. According to an example embodiment, the material matrix is a polymer. In one such embodiment, the polymer is either biostable or biodegradable.

In an embodiment of the invention, experimental data includes imaging data of T0 samples, imaging data at various time points, in vitro testing data, and in vivo testing data, amongst other examples.

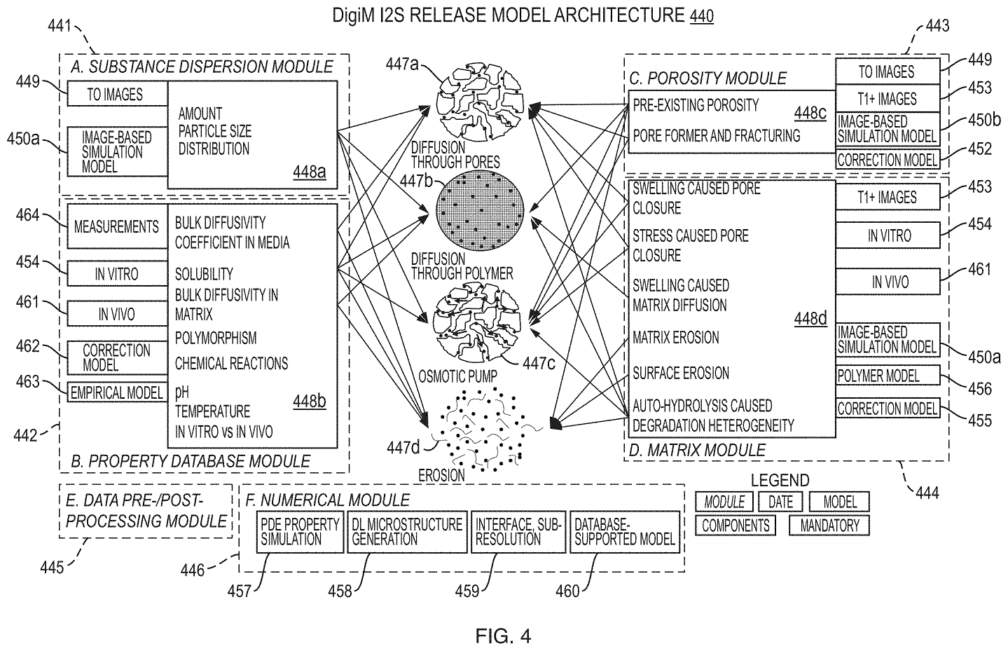

FIG. 4 illustrates a system 440 for determining a model predicting a release profile of a substance from a material matrix. The system 440 includes the modules 441-446 which are configured to implement functionality to iteratively predict release profiles. FIG. 4 also indicates release mechanisms 447a-d and the corresponding rate controlling parameters 448a-d. The rate controlled parameters 448a-d are the parameters that the release model captures in order to predict a release profile. As shown in FIG. 4, the rate-controlling parameters 448-d are grouped into four modules 441, 442, 443, and 444.

1.1 Substance Dispersion

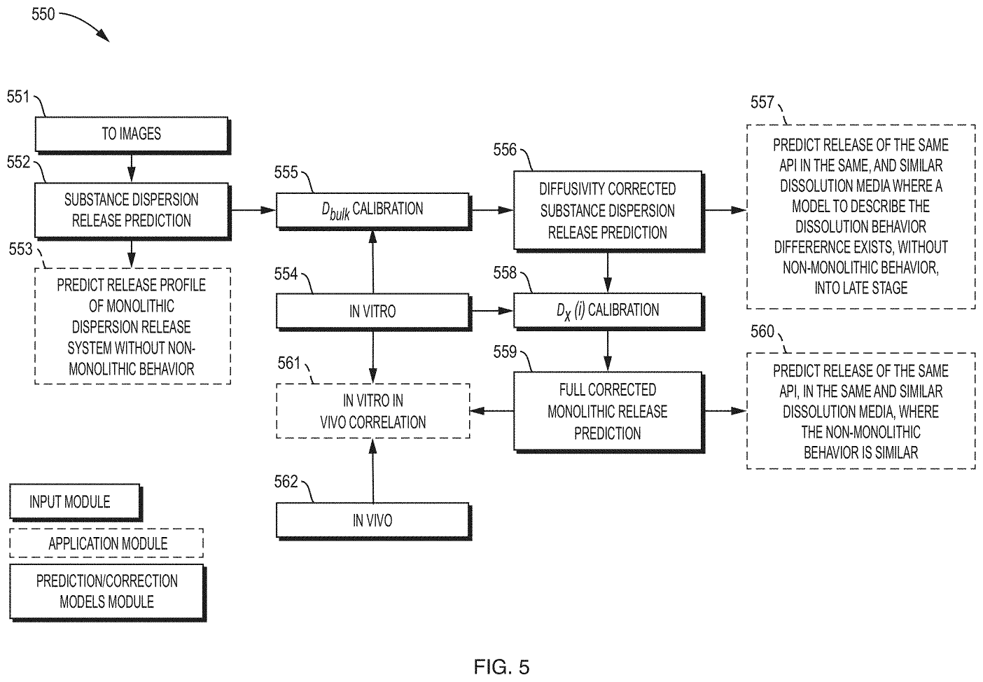

The substance dispersion module 441 captures the drug API network, including, for example the parameters 448a which include the amount of drug, the particle size, and its distribution in the release system. Moreover, additional substances, such as multiple APIs and functional excipients can be considered in the module 441 iteratively. For example, if there are two substances, the substance dispersion module 441 can consider substance1 only, substance2 only, substance 1 and 2 combined without interaction, and substance 1 and 2 combined with interaction. The substance dispersion module 441 utilizes T0 images 449, which are a data component shown in FIG. 4. The substance dispersion module 441 also includes an image-based simulation model component 450a shown in FIG. 4. The substance dispersion module 441 can implement a prediction mechanism, such as the prediction mechanism described in U.S. Pat. No. 11,081,212, to simulate the depletion of the substance layer by layer from the outside of the release system to the inside, while the matrix does not change during the release. This is further described hereinbelow in relation to the iterative workflow 550 described hereinbelow in relation to FIG. 5.

1.2 Property Database

Property database module 442 captures properties 448b of substance and media. Substance properties include bulk diffusivity coefficient in media, and solubility. Substance bulk diffusivity in matrix, which is both a substance property and a polymer property, can be considered by the property module 442. Likewise, any polymorph and crystallinity related considerations are included here. Properties associated with release media, such as pH dependence, temperature dependence, and other properties that are functions depending on in vitro 454, in vivo 461, and clinical release environment are also considered by the property database module 442. The module 442 can also integrate data and models, e.g. correction model 462 and empirical model 463, to factor in chemical reactions among substance(s), matrices, and release media. If measurement data 464 are available, the measurement data can be plugged-in directly as constants to other modules. Otherwise, these properties can be retrieved using a database, literature data, physiochemically constrained estimates, and correction methods provided by the module 442. When calibrated by available in vitro 454, in vivo 461, and clinical data, the calibrated properties can be recorded in the property database 442 which can be used for future blind predictions.

1.3 Porosity

Porosity module 443 captures the impact of porosity, including parameters 448c of pre-existing porosity and the formation of pores and fractures (possibly due to one of the additives being a pore-former or surfactant). The consideration of porosity uses T0 images 449 and an image-based simulation model (in pre-existing porosity case) 450b. The porosity module 443 may also utilize a correction model 452. T1+ images 453, if available, can be leveraged in the correction model 452.

1.4 Matrix

Matrix module 444 considers the impact of the matrix material, such as polymer, on release. A matrix influences release via swelling-caused pore closure, stress-caused pore closure (e.g., associated with manufacturing), swelling caused matrix diffusion (differentiated from the bulk diffusivity in matrix in the substance property module as this matrix diffusion coefficient changes over time), matrix erosion, surface erosion, and auto-hydrolysis caused degradation with potential heterogeneity. Several iterations may be necessary to consider polymer impact on release, depending on the release mechanism and availability of data. In one embodiment, in vitro data 454 can be used to derive a correction model 455 that incorporates all polymer behavior, without modeling each polymer behavior separately. In another embodiment, T1+ images 453 can be used to derive such a correction model 455. The in vitro data 454 and T1+ images 453 can also be used to derive a polymer model 456, which can be used to predict similar drug-polymer-porosity release systems. In another last case, a polymer is considered as a substance, and the components from substance dispersion module 441, i.e., T0 images 449 and Image-based simulation model 450a, can be invoked in a similar fashion. When in vivo data 461 is available, it can be employed in a similar manner as the in vitro data 454, resulting in a in vivo calibrated prediction model. In vitro-in vivo correlation (IVIVC) can be conducted using this method.

1.5 Data Pre-Processing and Post-Processing

The data pre-processing and data post-processing module 445 fits experimental data and simulation results.

1.6 Numerical Infrastructure

The numerical infrastructure module 446 includes several models 457-460. The property simulation model 457 operates on images governed by partial differential equations (PDE), such as effective diffusivity (Fick's law), permeability (Navier-Stokes), thermal conductivity (Fourier's law), and electrical conductivity (Ohm's law). The microstructure generation model 458 uses artificial intelligence methods to generate substance dispersion transport systems similar to a 3D image. The microstructure generation model 458 can also use artificial intelligence methods such as machine learning and deep learning, where previous imaging data, limited imaging data, and other physical testing data (e.g., laser diffraction-based particle size distribution) can be used to train deep learning models. The interface and sub-resolution model 459 considers substance-matrix interface and provides treatment when smaller scale phenomenon below current imaging resolution is needed. The database-supported model 460 provides functionality when numerical models need to interface with a database for both experimental data-based modeling and for artificial intelligence-based modeling.

2. An Iterative Method to Correct Release Prediction from T0 Imaging Using In Vitro Data, for Bulk D_{eff} and a Bulk Complex Behavior

D_{eff} is the effective diffusivity coefficient of a substance in a release media, when there is a porous matrix blockage. Hereinbelow, one embodiment of an iterative method 550 with three iteration steps to correct release prediction from T0 imaging data using in vitro data is described.

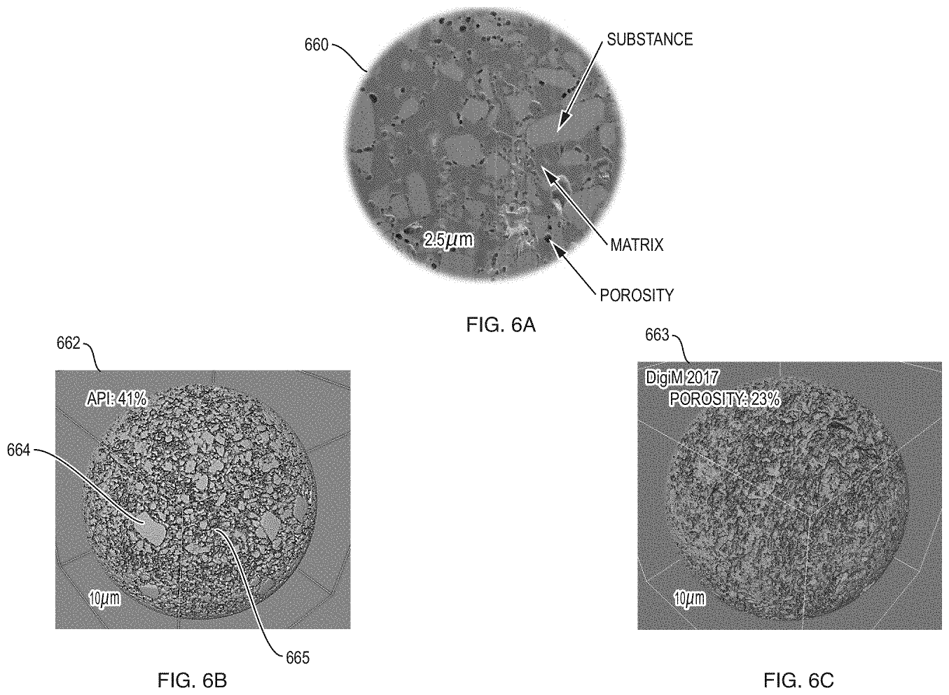

FIG. 5 shows an embodiment of an iterative method 550 to correct release prediction from T0 imaging data 551 using in vitro data 554. FIG. 5 also depicts uses of release profiles determined in the method 550 (e.g., the steps 557 and 560). To facilitate the discussion, FIGS. 6A-C shows a representative PLGA extended release microsphere sample, digitally transformed via focused ion beam scanning electron microscopy (FIB-SEM). FIG. 6A shows one cross section 660 of the 3D images acquired via FIB-SEM. The images can be acquired with any appropriate method. When 3D images are not available, the 3D digital representation can be reconstructed from 2D images or from an existing database. Via appropriate image segmentations on imaging data, e.g., the data 660, API drug particles, PLGA polymer matrix, and porosity can be segmented into their respective 3D networks. FIG. 6B shows the API network reconstructed in 3D 662. In FIG. 6B, the light shading, e.g., 664, indicates the substance (API), and the dark shading, e.g., 665 indicates the matrix. The 3D API network 662 depicted in FIG. 6B amounted to 41% of total sample volume. FIG. 6C shows the porosity network reconstructed in 3D 663. The porosity network segmented 663 amounts to 23% of the total sample volume. Representativeness has been considered using correlative imaging and statistical sampling approaches.

Returning to FIG. 5, below is a description of three iterations of the iterative method 550.

2.1 Iteration 1: Default Workflow

The first iteration encompasses a workflow supported by the functionality described in U.S. Pat. No. 11,081,212. The first iteration uses T0 imaging data 551 to predict the release of a drug substance (e.g., API 664 depicted in FIG. 6B) dispersed in polymer matrix (e.g., matrix 665 depicted in FIG. 6B) that is not changing over time. The first iteration implements the workflow 551 to 552 to 553.

In this workflow, a release profile of a release system with monolithic drug dispersion is predicted without the consideration of pore drug solubility, polymer, and porosity impact. Starting from T0 images (551), the release prediction module described in U.S. Pat. No. 11,081,212 (552) is used to predict a release profile of a monolithic dispersion release system (553) without complex behavior such as polymer change and solubility limitation.

2.2 Iteration 2: Calibration of D_{bulk}

D_{bulk} refers to the bulk diffusivity coefficient of the substance in dissolution media, without the blockage of a porous matrix. In the second iteration of the method 550, a correction associated with the uncertainty of D_{bulk}, which is a constant used in 552, is performed. Using spherical geometry (e.g., in extended release microsphere formulations) as an example, a release time interval is converted into physical time using modified Higuchi's model. The model is expressed in a spherical coordinate system, and rearranged for time conversion as shown in Equation 1:

$$T ( i ) = \frac{{R}^{2}}{2 ( \frac{{C}_{s}}{{C}_{0}} ) D ( i )} [ 1 - {( 1 - \frac{M ( i )}{{M}_{∞}} )}^{\frac{2}{3}} - \frac{2}{3} ( \frac{M ( i )}{{M}_{∞}} ) ] \qquad Eq . 1$$

In Equation 1 T is physical time, i is numerical time in percolation simulation (which determines the porous media domain where Fick's Second Law was solved), R is the radius of the microsphere sample, Cₛ is the total drug amount per unit volume of solution, C₀ is the initial drug amount per unit volume of solution, and

$$\frac{M ( i )}{{M}_{∞}}$$

is the fraction of drug released expressed in numerical time i. The numerical time is determined from percolation simulation with the assumption of no change in the solid drug particle density when the particles are still inside the microsphere.

It is noted that geometries other than sphere, and mathematical models other than Higuchi, can be employed in embodiments of the method 550.

D(i) is the effective diffusivity coefficient corresponding to the porous media ring at numerical time i, determined using direct numerical simulation of the portion of the sample where API has been released. In the simulation, actual fluid properties are normalized away, and only the effect of the porous medium network is considered. The final outcome of the simulation is expressed as Dₓ(i), a fraction of D_{bulk}, with a value between 0.0 and 1.0. The arithmetic relationship of D(i), D_{bulk}, and Dₓ(i) are expressed in Equation 2 below:

D(i)=D_{bulk}Dₓ(i) Eq. 2

D_{bulk} may be unknown, hence a reference literature value, such as glucose in water, is used in the first iteration described in section 2.1 (steps 551 to 552 to 553).

When physical testing data such as in vitro release profile is available, D_{bulk}, can be corrected using the following workflow: [(551+552)+554] to 555 to 556 to 557. In this case, T0 images (551) and a release prediction from a previous iteration (552) are enhanced by additional testing data, e.g., in vitro release profile, (554). D_{bulk} can be calibrated using the appropriate sections in the in vitro release data (554), where it matches the best with release prediction results 553 during the initial phase of release. With this calibration, the substance release prediction is corrected with a more realistic D_{bulk} (555). After this correction, the release model can predict release of the same API in the same, and similar dissolution media where a model to describe the dissolution behavior difference exists, without complex behavior, into late stage (557).

In addition to D_{bulk} correction, this iteration can also reveal which phase of the release complies with the monolithic behavior, and which phase deviates due to polymer or solubility complexities. For example, if a release system has an initial burst, the initial burst rate most accurately corresponds to the effective diffusivity coefficient of the API in the release media.

Further, it is noted that the in vitro release profile (554) is not the only data that can be used to make D_{bulk} correction.

2.3 Iteration 3: Simple Calibration of Dₓ(i)

In Equation 2, Dₓ(i) is computed based on the monolithic drug release in iteration 1 (Iteration 1 described in section 2.1). When polymer, porosity, and drug-polymer interaction complicate the release, it is no longer accurate to compute Dₓ(i) using only the drug network. A Dₓ(i) correction model can then be derived directly using in vitro data 554. The third iteration of the method 550 utilizes the workflow [(551+556)+554] to 558 to 559 to 560 to provide such functionality.

In this case, T0 images (551) and release prediction from a previous iteration (556) are enhanced by available testing data, e.g., in vitro release profile the second time to correct Dₓ(i). Through a least square fitting between the predicted release profile (556) and in vitro release profile (554), a Dₓ(i) calibration model (558) is derived. This Dₓ(i) calibration model 558 can then be used, in conjunction with previously corrected models 552 and 556, to get fully correct monolithic release prediction (559). This prediction model 559 can then be used to predict release systems with the same API, in the same and similar dissolution media, where the complex behavior is similar (560). One example for such an applicable prediction is for a sample formulation with different drug loading, while everything else remains the same.

2.4 Software Architecture and Future Iterations

The correction from section 2.3 leverages the full in vitro release profile, while lumping the highly complex polymer change and solubility limit into one empirical correction model 559. When additional data is available, e.g., the in vivo release profile 562, further iterations can continue to use the additional data (in vivo data 562) along with the in vitro data 554 to correct the previous model 559. One such embodiment uses the fully corrected monolithic release prediction model 559, in vitro data 554, and in vivo data 562 to improve the in vitro in vivo correlation (IVIVC) 561.

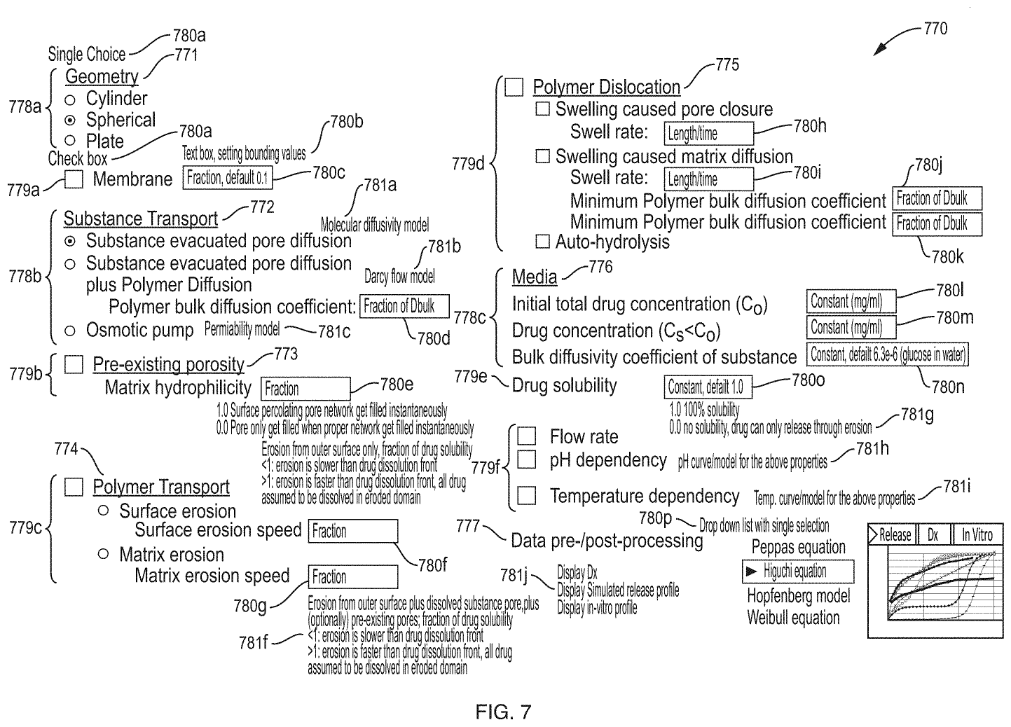

FIG. 7 shows an embodiment of a graphical user interface (GUI) design 770 that can support embodiments and the aforementioned future iterations. In FIG. 7 the GUI elements 778a-c show user inputs that may be mandatory in an embodiment and the GUI elements 779e-f show user inputs that may be optional in an embodiment. The text 780a-p describes expected user input or user choice. The text 781a-j briefly describes the documentation of the parameters.

The interface 770 of FIG. 7 includes the input modules 771-777.

The geometry module 771 allows the user to select the geometry of the release system. It can be cylindrical, spherical, or a plate. A membrane coating with certain thickness can also be indicated via the geometry input module 771. An embodiment processes input image data in accordance with the geometry selection.

The substance transport module 772 allows a user to choose a substance transport type. The aforementioned three iterations described in relation to FIG. 5 uses “substance evacuated pore diffusion” option, where the diffusion of the API dominates.

In an example future iteration, if there is reason to believe that the drug might also diffuse through the polymer matrix, then the option “substance evacuated pore diffusion plus polymer diffusion” can be selected via the module 772. A different transport model, such as multiphase Darcy model can then be used. The module 772 allows a user to specify the bulk diffusion coefficient of the API transport through the polymer, as a fraction of the diffusion coefficient of the API transport through bulk fluid (no porous media nor polymer). Tortuosity of the polymer domain can be considered. The iterations described in sections 2.1, 2.2 and 2.3 can be applied on the prediction of this substance transport model.

The pre-existing porosity module 773 allows a user to indicate the impact of porosity. When the polymer matrix is hydrophilic, fluid does not get into the porosity prior to API dissolution, hence porosity exposure can only happen when the adjacent API is dissolved. Hence, only the porosity inside the released-drug zone contributes to the diffusion of the remaining drug. If the polymer system is hydrophobic, the porosity will be saturated with water prior to API release. The API release will be accelerated as the API interior to the sample, when in contact with water saturated pores, will be dissolved and released through the porosity network. When drug loading is not high enough to form a percolation network, the diffusion through water filled pores will be the primary network of release. If the porosity is low and does not form a percolation network, then its contribution to release is small, and only felt when it is inside the released-drug zone.

The polymer transport module 774 allows a user to indicate whether surface erosion or matrix erosion should be considered.

The polymer dislocation module 775 is a module that allows a user to indicate whether swelling caused pore closure, swelling caused matrix diffusion, or auto-hydrolysis which leads to polymer dislocation heterogeneity should be considered.

The media module 776 provides the necessary controls on drug concentration, bulk diffusivity coefficient, and drug solubility.

A supporting data pre-/post-processing module 777 provides the visualization functions, time conversion, and data fitting.

Embodiments and the iterations may consider, amongst other examples: in-vitro in-vivo correlation when in vivo data is available, iterative correction using T1+ imaging data, iterative correction using compounding polymer models, and iterative correction using literature/empirical models.

With the help of the iterative methods and software design described herein, complex release systems can be investigated for better design, and predicted to reduce time and cost for research, development, and manufacturing.

Computer Support

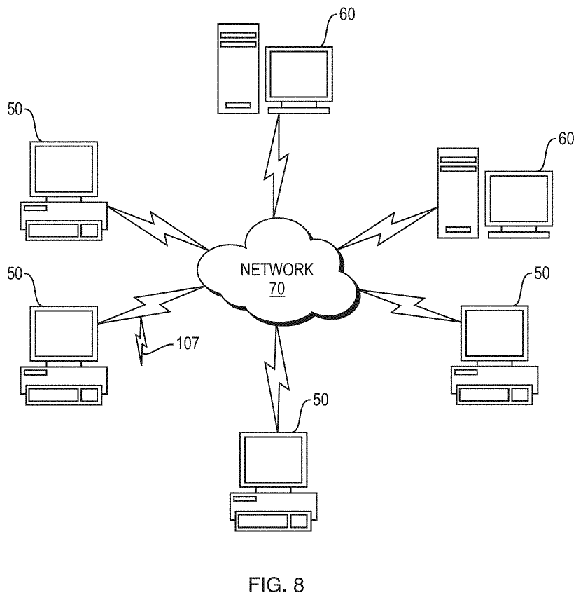

FIG. 8 illustrates a computer network or similar digital processing environment in which embodiments of the present invention may be implemented. Client computer(s)/device(s) 50 and server computer(s) 60 provide processing, storage, and input/output devices executing application programs and the like. The client computer(s)/device(s) 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

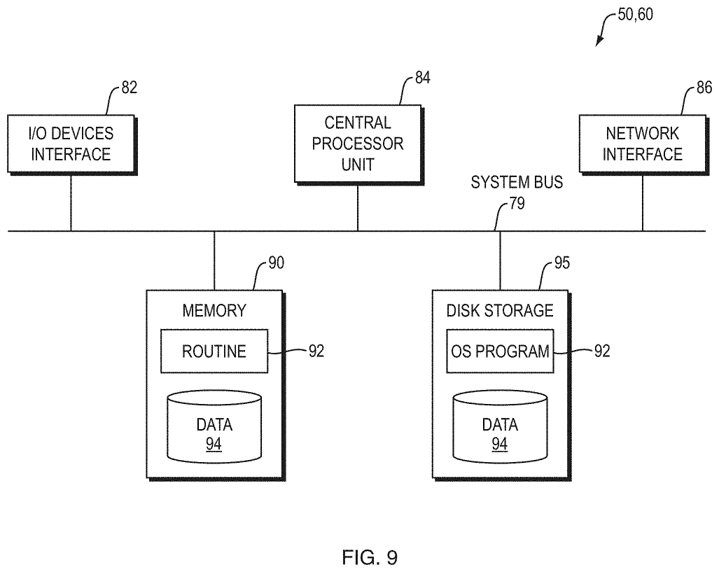

FIG. 9 is a diagram of an example internal structure of a computer (e.g., client processor/device 50 or server computers 60) in the computer system of FIG. 8. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) and enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to other various devices attached to a network (e.g., network 70 of FIG. 8). Memory 90 provides volatile storage for computer software instructions 92 and data 94 used to implement embodiments of the present invention (e.g., method 220, method 322, system 440, method 550, and interface 770, amongst others detailed herein). Disk storage 95 provides non-volatile storage for the computer software instructions 92 and the data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory, computer-readable medium (e.g., a removable storage medium such as one or more internal hard drives, external hard drives, DVD-ROMs, CD-ROMs, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other networks. In one embodiment, the propagated signal is a signal that is transmitted over the propagation medium over a period of time, such as the instructions for a software application sent in packets over a network over a period of milliseconds, seconds, minutes, or longer.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

### REFERENCES

Arifin, Y., Lee, L. Y., Wang, C., 2006. Mathematical modeling and simulation of drug release from microspheres: implications to drug delivery systems. Adv. Drug Deliv. Rev. 58, 1274-1325.
Barat, A., Crane, M., Ruskin, H. J., 2008. Quantitative multi-agent models for simulating protein release from PLGA bioerodible nano- and microspheres. J. Pharm. Biomed. Anal. 48, 361-368.
Batycky, R. P., Hanes, J., Langer, R., Edwards, D. A., 1997. A theoretical model of erosion and macromolecular drug release from biodegrading microspheres. J. Pharm. Sci. 86, 1464-1477.
Bertoldi, C., Zaffe, D., Consolo, U., 2008. Polylactide/polyglycolide copolymer in bone defect healing in humans. Biomaterials 29, 1817-1823.
Ehtezazi, T., Washington, C., 2000. Controlled release of macromolecules form PLA microspheres: using porous structure topology. J. Control. Release 68, 361-372.
Feng, L., Qi, X. R., Zhou, X. J., Maitani, Y., Wang, S. C., Jiang, Y., Nagai, T., 2006. Pharmaceutical and immunological evaluation of a single-dose hepatitis B vaccine using PLGA microspheres. J. Control. Release 112, 35-42.
Fredenberga S., Wahlgrenb M., Reslowc M., Axelssona A. The mechanisms of drug release in poly(lactic-co-glycolic acid)-based drug delivery systems—A review, International Journal of Pharmaceutics 415 (2011) 34-52.
Guse, C., Koennings, S., Blunk, T., Siepmann, J., Goepferich, A., 2006a. Programmable implants—from pulsatile to controlled release. Int. J. Pharm. 314, 161-169.
Jiang, W., Gupta, R. K., Deshpande, M. C., Schwendeman, S. P., 2005. Biodegradable poly(lactic-co-glycolic acid) microparticles for injectable delivery of vaccine antigens. Adv. Drug Deliv. Rev. 57, 391-410.
Mollo, A. R., Corrigan, O. I., 2003. Effect of poly-hydroxy aliphatic ester polymer type on amoxicillin release from cylindrical compacts. Int. J. Pharm. 268, 71-79.
Murty, S. B., Wei, Q., Thanoo, B. C., DeLuca, P. P., 2004. In vivo release kinetics of octreotide acetate from experimental polymeric microsphere formulations using oil/water and oil/oil processes. AAPS Pharm. Sci. Tech. 5 (article 49).
Närhi, M., Nordstrom, K., 2005. Manufacturing, regulatory and commercial challenges of biopharmaceuticals production: a Finnish perspective. Eur. J. Pharm. Biopharm. 59, 397-405.
Oh, S. H., Lee, J. H., 2007. Fabrication and characterization of hydrophilized porous PLGA nerve guide conduits by a modified immersion precipitation method. J. Biomed. Mater. Res. 80A, 530-538.
Pisal, D. S., Kosloski, M. P., Baly-iyer, S. V., 2010. Delivery of therapeutic proteins. J. Pharm. Sci. 99, 2557-2575.
Siepmann, J., Gopferich, A., 2001. Mathematical modelling of bioerodible, polymeric drug delivery systems. Adv. Drug. Deliv. Rev. 48, 229-247.
Siepmann, J., Siepmann, F., 2008. Mathematical modeling of drug delivery. Int. J. Pharm. 364, 328-343.
Susanne Fredenberga, Marie Wahlgrenb, Mats Reslowc, Anders Axelssona. The mechanisms of drug release in poly(lactic-co-glycolic acid)-based drug delivery systems—A review, International Journal of Pharmaceutics 415 (2011) 34-52.
Raiche, A. T., Puleo, D. A., 2006. Modulated release of bioactive protein from multilayered blended PLGA coatings. Int. J. Pharm. 311, 40-49.
Wang, X., Sui, S., Yan, Y., Zhang, R., 2010. Design and fabrication of PLGA sandwiched cell/fibrin constructs for complex organ regeneration. J. Bioactive Compatible Polym. 25, 229-240.
Wiscke, C., Schwendeman, S. P., 2008. Principles of encapsulating hydrophobic drugs in PLA/PLGA microparticles. Int. J. Pharm. 364, 298-327.
Zhang S. System and method for computing drug controlled release performance using images, U.S. Patent App., Pub. No. 2019/0108322 A1, published Apr. 11, 2019.

What is claimed is:

1. A computer-implemented method of determining a model predicting a release profile of a substance from a material matrix, the method comprising:
constructing a simulation model of a release system based on imaging data of the release system, wherein the release system comprises a material matrix and a substance dispersed in the material matrix; and
determining a model predicting a release profile of the substance from the material matrix by:
until a given simulation-based release profile matches release system characteristic data, iteratively: (i) modifying parameters of the constructed simulation model based upon the release system characteristic data to correct a transport coefficient of the release system and (ii) performing a simulation of the release system using the constructed simulation model with the modified parameters to generate a simulation-based release profile; and
setting the constructed simulation model with the modified parameters used in performing the simulation to generate the given simulation-based release profile, as the model predicting the release profile of the substance from the material matrix.

2. The method of claim 1 wherein modifying the parameters of the constructed simulation model based upon the release system characteristic data comprises:
modifying parameters of the constructed simulation model affecting the transport coefficient.

3. The method of claim 1 where the imaging data is of a representative sample of the release system and wherein the simulation model is constructed based on both the imaging data of the representative sample of the release system and known parameters approximating a release mechanism of the release system.

4. The method of claim 1 wherein determining the model predicting the release profile of the substance from the material matrix comprises:
in an iteration:
creating a first modified model of the release system by modifying, in accordance with the release system characteristic data, given parameters of the constructed simulation model to modify the transport coefficient due to a difference between actual diffusivity of the substance and diffusivity in the given parameters; and
performing a simulation using the first modified model to determine a first simulation-based release profile, wherein the first simulation-based release profile is the given simulation-based release profile that matches the release system characteristic data.

5. The method of claim 1 wherein determining the model predicting the release profile of the substance from the material matrix comprises:
in a first iteration:
creating a first modified model of the release system by modifying, in accordance with the release system characteristic data, given parameters of the constructed simulation model to modify the transport coefficient due to a difference between actual diffusivity of the substance and diffusivity in the given parameters; and
performing a simulation using the first modified model to determine a first simulation-based release profile, wherein the first simulation-based release profile does not match the release system characteristic data;
in a second iteration:
creating a second modified model of the release system by modifying, in accordance with the release system characteristic data, given parameters of the first modified model to correct changes to the transport coefficient due to variability in diffusivity of the substance over time caused by changes of the material matrix during the release; and
performing a simulation using the second modified model to determine a second simulation-based release profile, wherein the second simulation-based release profile is the given simulation-based release profile matching the release system characteristic data.

6. The method of claim 1 where the simulation model is constructed based upon both the imaging data and known parameters that approximate a release mechanism of the release system and the release system characteristic data comprises in vitro release system characteristic data and, wherein the determining a model predicting the release profile of the substance from the material matrix comprises:
until the given simulation-based release profile matches the in vitro release system characteristic data, iteratively (i) modifying the parameters of the constructed simulation model affecting the transport coefficient in accordance with the in vitro release system characteristic data and (ii) performing a simulation of the release system using the constructed simulation model with the modified parameters to generate a simulation-based release profile.

7. The method of claim 1 further comprising:
determining the given simulation-based release profile matches the release system characteristic data by performing a least squares fitting between the given simulation-based release profile and a release profile indicated by the release system characteristic data.

8. The method of claim 1 wherein the release system characteristic data includes at least one of: images of the release system at multiple points in time; in vitro data of the release system; in vivo data of the release system; a polymer model; an empirical model; an indication of geometry of the release system being cylindrical, spherical, or plate; membrane coating dimensions; an indication of substance transport being substance evacuated pore diffusion, substance evacuated pore diffusion plus polymer diffusion, or osmotic pump; an indication of pre-existing porosity; an indication of polymer transport as surface erosion or matrix erosion; an indication of polymer dislocation as swelling caused pore closure, swelling caused matrix diffusion, or auto-hydrolysis; drug concentration; bulk diffusivity; drug solubility; flow rate; pH dependency; and temperature dependency.

9. The method of claim 1 wherein the substance is at least one of:
a pharmaceutical ingredient;
an insecticide; and
an anti-corrosion agent.

10. The method of claim 1 wherein the material matrix is a polymer.

11. The method of claim 10 wherein the polymer is either biostable or biodegradable.

12. The method of claim 1 wherein constructing the simulation model of the release system based on the imaging data of the release system comprises:
determining at least one image-derived release system characteristic from the imaging data; and
updating a parameter of the simulation model of the release system to correspond to the determined at least one image-derived release system characteristic.

13. The method of claim 12 wherein the at least one image-derived release system characteristic includes: size of the substance, amount of the substance, location of the substance in the release system, size of the material matrix, amount of the material matrix, location of the material matrix, porosity of the material matrix, pore size of the material matrix, location of pores, fracture size, fracture location, size of an additive in the release system, amount of the additive, location of the additive in the release system, and evolution of the at least one image-derived release system characteristic over time.

14. The method of claim 1 wherein the transport coefficient indicates at least one of:
diffusivity of the substance through the release system;
diffusivity of the substance over time caused by changes of the release system during the release;
permeability of the substance through the release system; and
permeability of the substance over time caused by changes of the release system during the release.

15. The method of claim 14 wherein the changes of the release system include at least one of: phase transformation of the substance; phase transformation of the matrix; polymorph transformation of the substance; polymorph transformation of the matrix; degradation of the matrix; surface erosion of the matrix; bulk erosion of the matrix; matrix swelling; matrix deformation; matrix dislocation; pore forming; pore closure; fractures; hydrolysis; relocation of the substance; relocation of the matrix, aggregation of the substance; aggregation of the matrix; dispersion of the substance; and dispersion of the matrix.

16. A computer system for determining a model predicting a release profile of a substance from a material matrix, the computer system comprising:
a processor; and
a memory with computer code instructions stored thereon, the processor and the memory, with the computer code instructions, being configured to cause the system to:
construct a simulation model of a release system based on imaging data of the release system, wherein the release system comprises a material matrix and a substance dispersed in the material matrix; and
determine a model predicting a release profile of the substance from the material matrix by:
until a given simulation-based release profile matches release system characteristic data, iteratively: (i) modifying parameters of the constructed simulation model based upon the release system characteristic data to correct a transport coefficient of the release system and (ii) performing a simulation of the release system using the constructed simulation model with the modified parameters to generate a simulation-based release profile; and
setting the constructed simulation model with the modified parameters used in performing the simulation to generate the given simulation-based release profile, as the model predicting the release profile of the substance from the material matrix.

17. The system of claim 16 wherein, in determining the model predicting the release profile of the substance from the material matrix, the processor and the memory, with the computer code instructions, are further configured to cause the system to:
in an iteration:
create a first modified model of the release system by modifying, in accordance with the release system characteristic data, given parameters of the constructed simulation model to modify the transport coefficient due to a difference between actual diffusivity of the substance and diffusivity in the given parameters; and
perform a simulation using the first modified model to determine a first simulation-based release profile, wherein the first simulation-based release profile is the given simulation-based release profile that matches the release system characteristic data.

18. The system of claim 16 wherein, in determining the model predicting the release profile of the substance from the material matrix, the processor and the memory, with the computer code instructions, are further configured to cause the system to:
in a first iteration:
create a first modified model of the release system by modifying, in accordance with the release system characteristic data, given parameters of the constructed simulation model to modify the transport coefficient due to a difference between actual diffusivity of the substance and diffusivity in the given parameters; and
perform a simulation using the first modified model to determine a first simulation-based release profile, wherein the first simulation-based release profile does not match the release system characteristic data;
in a second iteration:
create a second modified model of the release system by modifying, in accordance with the release system characteristic data, given parameters of the first modified model to correct changes to the transport coefficient due to variability in diffusivity of the substance over time caused by changes of the material matrix during the release; and
perform a simulation using the second modified model to determine a second simulation-based release profile, wherein the second simulation-based release profile is the given simulation-based release profile matching the release system characteristic data.

19. The system of claim 16 where the simulation model is constructed based upon both the imaging data and known parameters that approximate a release mechanism of the release system and the release system characteristic data comprises in vitro release system characteristic data and, wherein, in determining the model predicting the release profile of the substance from the material matrix, the processor and the memory, with the computer code instructions, are further configured to cause the system to:
until the given simulation-based release profile matches the in vitro release system characteristic data, iterate (i) modifying the parameters of the constructed simulation model affecting the transport coefficient in accordance with the in vitro release system characteristic data and (ii) performing a simulation of the release system using the constructed simulation model with the modified parameters to generate a simulation-based release profile.

20. A computer program product for determining a model predicting a release profile of a substance from a material matrix, the computer program product comprising:
one or more non-transitory computer-readable storage devices and program instructions stored on at least one of the one or more storage devices, the program instructions, when loaded and executed by a processor, cause an apparatus associated with the processor to:
construct a simulation model of a release system based on imaging data of the release system, wherein the release system comprises a material matrix and a substance dispersed in the material matrix; and
determine a model predicting a release profile of the substance from the material matrix by:
until a given simulation-based release profile matches release system characteristic data, iteratively: (i) modifying parameters of the constructed simulation model based upon the release system characteristic data to correct a transport coefficient of the release system and (ii) performing a simulation of the release system using the constructed simulation model with the modified parameters to generate a simulation-based release profile; and
setting the constructed simulation model with the modified parameters used in performing the simulation to generate the given simulation-based release profile, as the model predicting the release profile of the substance from the material matrix.
